(19) United States
(12) Patent Application Publication
(10) Pub. No.: US 2018/0361175 A1
(43) Pub. Date: Dec. 20, 2018

(54) MULTICOMPONENT BLEACHING SYSTEM

(71) Applicant: Colgate-Palmolive Company, New York, NY (US)

(72) Inventors: Guofeng XU, Plainsboro, NJ (US); Stacey LAVENDER, Chesterfield, NJ (US); LaTonya KILPATRICK-LIVERMAN, Princeton, NJ (US); Scott DEMAREST, Basking Ridge, NJ (US); Cajetan DOGO-ISONAGIE, Highland Park, NJ (US)

(73) Assignee: Colgate-Palmolive Company, New York, NY (US)

(21) Appl. No.: 16/062,144

(22) PCT Filed: Dec. 17, 2015
(86) PCT No.: PCT/US2015/066294

§ 371 Date: Jun. 14, 2018

(51) Int. Cl. A61Q 11/02; A61K 8/22; B65D 81/32; A61K 8/38

(52) U.S. Cl. CPC A61Q 11/02; A61K 8/22; A61K 8/466; A61K 8/38; B65D 81/3266

(57) ABSTRACT

The invention provides packages for storing and dispensing multi-component tooth whitening formulations, wherein the contents of the separate components are mixed just prior to use. Particular multi-component tooth whitening formulations using this principle and methods of use thereof are also provided.

### FIELD OF THE INVENTION

The present invention relates to a single use or multiple-use, multi-component system and device for whitening teeth, which provides a stable strong whitening agent that is activated just prior to use.

### BACKGROUND

It is frequently desirable to keep formulation components separate prior to use, for example, because the components may be too unstable for long-term storage if combined. It is desirable in such cases to be able to mix the formulation components at the point of use in an efficient and simple way, and also to apply the resulting mixed formulation to difficult-to-reach places.

One example of a composition where it may be desirable to keep formulation components separate is tooth-whitening compositions comprising reactive ingredients such as peroxides, peroxyacids, persulfates or their precursors. For example, one may want to combine A+B or A+B+C to obtain an unstable bleach X, but keep A and B separate up to that point. The difficulty arises in that during use the mixing must be rapid, and diffusion of the bleaching agent, X, to the tooth surface must be efficient. A common example of these dual component compositions is one where one component is the bleaching agent or bleaching agent precursor, and the other is an ingredient needed to activate the bleaching agent or bleaching agent precursor. By keeping the bleaching agent separate from the activating agent, the shelf life of the product is greatly extended. At time of use, the agents become combined and the bleaching agent is activated (or fully activated, if it has nominal activity before mixing). Another example of a dual component composition in one where one component is the bleaching agent, and the second component includes something that would inactivate the bleaching agent (but is otherwise beneficial for the composition). In this situation, the two components cannot be stored together in a single composition because this would result in a very short shelf life for the product. However, if the two components are kept separate and only mixed upon use, then the bleaching agent will remain sufficiently active to complete at least one instance of use. A similar situation arises where the active whitening agent is best maintained in a hydrophobic environment, but where water is necessary or beneficial to the activation of the whitening agent.

In addition, in the field of tooth-whitening compositions, a common problem is that the usual consumer formulations (e.g., adhesive strips) do not allow the user to apply tooth-whitening compound to hard-to-reach sites, for example the edges or surfaces between adjacent teeth.

There is thus a need for products that maintain bleaching agent stability for long term storage with rapid activation upon consumer use, and efficient point of use mixing.

### BRIEF SUMMARY

In one aspect, the present disclosure provides a single use, multi-chamber system, wherein at least one chamber contains a bleaching agent or bleaching agent precursor, and the two or more chambers contain ingredients that are in some way incompatible with each other for storage.

In some embodiments, the chambers are separated by a frangible or tearable barrier, such that upon squeezing one chamber, the barrier breaks and the components of the chambers can mix, to form a solution, emulsion, suspension or extrudable gel, which can be dispensed through an outlet in the second chamber, wherein the contents of the chambers, upon mixing, provide an effective bleaching composition. In these embodiments, the frangible or tearable barrier must be broken to allow mixing of the components and the barrier also serves to protect the contents of each chamber (e.g., from contamination, spillage).

In some embodiments, each chamber has an exit channel whereby the contents of each chamber may be extruded through the exit channel where the contents will become mixed to form a solution, emulsion, suspension or extrudable gel, which can be dispensed through a common outlet, such that the contents of the chambers, upon mixing, provide an effective bleaching composition. In some embodiments, the exit channels of the chambers extrude their contents into a common mixing tip for dispensing. In some embodiments there is a frangible or tearable barrier sealing the opening of the exit channels. In some embodiments the chambers are contained within the barrel of a syringe such that when the plunger of the syringe is depressed the contents of each chamber are extruded for mixing and dispensing.

In one embodiment, there are at least two chambers, and the first chamber contains a bleaching agent solution, gel or powder (e.g., anhydrous potassium monoperoxysulfate), and the second chamber contains an aqueous solution or gel (e.g., of a particular pH, for example, pH 5-8). Potassium monoperoxysulfate is a powerful oxidizing agent that has seen limited use in dental whitening compositions because of its instability in aqueous solution, especially in aqueous solution near or above neutral pH. While potassium monoperoxysulfate is somewhat stable in aqueous solutions at acidic pH, it is more stable either as a solid or in an anhydrous formulation, and it is most active as an oxidizing agent (and tooth whitening agent) in an aqueous solution at pH 5-8.

In another embodiment, there are at least two chambers, and the first chamber contains a bleaching agent precursor solution or gel (e.g., sodium nonanoylbenzenesulfonate) and the second chamber contains an activating agent solution or gel (e.g. hydrogen peroxide). Upon mixing the contents of the chambers, the active bleaching agent peroxynonanoic acid is formed.

In certain embodiments, additional chambers are provided, such as, for example, a third chamber containing a secondary whitening agent (e.g., urea peroxide) or a whitening enhancer (e.g., ammonium persulfate).

In some embodiments, the device comes with an applicator that can be used to help mix the components of the chambers, and having mixed them, can be used to help apply to active formulation to the teeth, and in particular, to hard-to-reach areas of the teeth. In certain embodiments, the device may be designed such that the applicator can be used to puncture the frangible barrier between the compartments in order to initiate mixing of the components. In other embodiments, the applicator has a tip (e.g. a brush tip) that is designed to enable application of the whitening agent to particularly hard-to-reach areas of the teeth.

In another aspect, the present disclosure provides a multiple-use device comprising two or more chambers with adjacent exit-channels that extrude the contents into a mixing tip. The mixing tip causes mixing and activation of the incompatible ingredients, as described above, resulting in dispensing of an active whitening composition from the mixing tip, which can then be applied to the teeth. Because the contents of the two chambers only mix in the mixing tip, the package can be reused simply by fitting a cap or other seal to the end of the mixing tip after initial use. In some embodiments the chambers are contained within the barrel of a syringe such that when the plunger of the syringe is depressed the contents of each chamber are extruded for mixing and dispensing.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 depicts one embodiment of the invention which is a two-chambered package in accordance with the invention, the package being heat-sealed about the perimeter (1), and having a first chamber (2) which contains a first component and a second chamber (4) containing a second component, separated by a frangible seal (3), such that when the first chamber (2) is squeezed, the frangible seal (3) ruptures and the first component flows into the second chamber (4) and mixes with the second component, which resulting mixture can then be dispensed by breaking the scored edge (5) to allow the mixture to flow or be squeezed out of the nozzle (6).

FIG. 2 depicts another embodiment of the invention, permitting mixing of components just prior to use, as described for FIG. 1, but utilizing a three-chambered package having a nozzle which can be opened by the consumer for dispensing product. In this embodiment, the package comprises a first chamber (7), a second chamber (8), a third chamber (9), the chambers being separated by frangible seals (10), and a nozzle with a break-away tip (11) to dispense the materials after mixing.

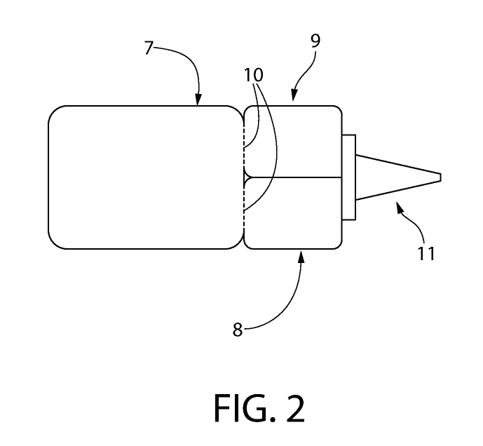

FIGS. 3A (top view) and 3B (side view) depict another embodiment of a two-chamber package similar to FIG. 1, except that the supplied applicator (15) is used to rupture the frangible seals (14). The applicator can then be used to help with mixing of the components of chamber (12) and (13) prior to dispensing the mixture through the nozzle (16).

FIG. 4 depicts a second aspect of the disclosure, a two-chamber single or multiple-use package. The two chambers (17) and (18) lie adjacent to each other with their exit channels adjacent and exiting via a common neck (19). Fitted over the neck is a removable mixing tip (20), and covering the exit channel opening in the neck is an optional frangible barrier (21).

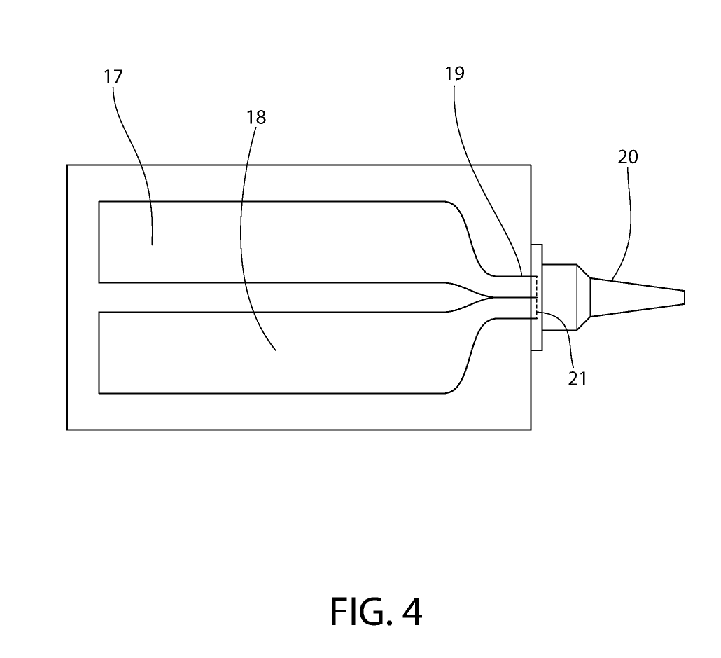

### DETAILED DESCRIPTION

The following description of the preferred embodiment(s) is merely exemplary in nature and is in no way intended to limit the invention, its application, or uses.

In one aspect, the invention provides a single use package for an oral care product which comprises multiple chambers (e.g., two or three or more) and is designed to keep the ingredients in each chamber separate and non-reactive until the point of use. For example, this invention provides a chemically stable structural package design which allows rapid mixing immediately prior to use of the components that are incompatible to long term storage together. The contents of the container are dispensed via an opening means, e.g., through a nozzle or exit channel with a removable cap or plug or which becomes functional when a preferentially scored section of the container is broken off by the consumer or when a frangible seal is broken, permitting clean and convenient dispensing of product through a shaped nozzle.

In a second aspect, the invention provides a multiple use package for an oral care product which also comprises multiple chambers and is designed to keep the ingredients in each chamber separate and non-reactive until the point of use.: For example, this aspect provides a chemically stable structural package design which allows rapid mixing immediately prior to use of the components that are incompatible to long term storage together, the mixing taking place in a mixing tip.

In some embodiments, the single use or multiple use package is a multi-chamber syringe, such that when the plunger of the syringe is depressed the contents of each chamber are extruded for mixing and dispensing. Each chamber may include an exit channel such that the exit channels dispense into a common nozzle, mixing chamber, or through an attached or detachable mixing tip.

In some embodiments, each chamber has the capacity to store, e.g., 0.1-30 grams of an ingredient. The oral care product is a tooth whitening product providing a total quantity of product delivered from all chambers, e.g., between 1.0 to 5.0 grams, for example 1-2 grams to provide the intended benefit.

In one embodiment, the package is manufactured using a thermoforming process of at least two flexible films with a thickness of 50 microns to 500 microns and preferably about 300 microns thick. The two films may be opaque, translucent or transparent and can be any combination when assembled in the thermoforming process. Both materials provide water vapor barrier characteristics, e.g., with less than 3% moisture loss over a three year time frame, e.g., less than 1% moisture loss over the same period. The films also provide flavor barrier. The flavor loss can be determined both by gas chromatography and by organoleptic evaluation.

The films are chemically resistant to the materials comprised therein, for example, to the oxidizing agents that comprise the tooth whitening composition. For example, in one embodiment, they are resistant to 0.1 to 30% by weight hydrogen peroxide solution or gel, or to 0.1 to 30% by weight of potassium monoperoxysulfate solution or gel, or to 0.1 to 30% by weight of peroxynonanoic acid solution or gel, e.g., 0.5 to 20% by weight, or 1 to 10% by weight, of the these agents.

In one embodiment, one of the two flexible materials is a polymeric laminate and the inner layer of the laminate has been selected to bond with the first flexible material but will delaminate when pressure is manually applied to the chamber with a frangible seal. The force required to break the seal is manually applied and can vary between 2 inch-1 bf and 5 inch-1 bf.

Where chambers are separated by a frangible seal, when the seals between the compartments are broken, the ingredients in each chamber will come into intimate contact with each other. The consumer may promote mixing of the ingredients by breaking the frangible seal between two chambers then mixing the contents by massaging the package over the two chambers. Alternatively, in some embodiments, the applicator can be used to help mix the contents of the chambers after breaking two frangible seals. Once fully mixed, the complete composition may be dispensed into a tray or other receptacle then applied to the teeth, or directly applied to the teeth, optionally using the applicator. The composition is left on the teeth for a period of time of 15 minutes to 45 minutes and provides an effective whitening benefit.

Exemplary embodiments of the invention thus include, for example, packages, oral care compositions, and methods of whitening teeth, e.g.:
1. Package 1, a single-use package comprising a material (e.g., deformable or non-deformable material) configured to form at least two sealed chambers, the package having
   (i) a first chamber, a second chamber, and optionally additional chambers, wherein the chambers are optionally separated by one or more barriers which are frangible or tearable, wherein the contents of the two chambers are incompatible with each for storage, wherein when one or more of the barriers are broken (e.g., as a result of squeezing the chamber or by puncturing the barrier or by removing the barrier), the contents of the chambers mix or can be mixed and interact e.g. chemically react) to form an active tooth-whitening composition, and
   (ii) an opening means, for example a scored region, cap or plug, or one or more exit channels with a frangible barrier, cap or plug, which allows opening of the package, mixing of the components of the chambers and providing an outlet through which the active composition can be dispensed after mixing of the components.
1.1. Package 1 wherein at least one first chamber contains a bleaching agent (e.g., a substantially anhydrous persulfate salt) or bleaching agent precursor (e.g., an alkyloxybenzenesulfonic acid or alkyloxybenzenesulfonic acid salt), and at least one second chamber contains an activating agent, e.g., an agent that activates a bleaching agent precursor or promotes or enhances the activity of a bleaching agent, for example, an aqueous solution or a peroxide source, or one or more ingredients incompatible with the contents of the first chamber, e.g., an aqueous solution or a peroxide source.
1.2. Package 1 or 1.1, wherein a first chamber contains a bleaching agent (e.g., a substantially anhydrous persulfate salt) or bleaching agent precursor (e.g., an alkyloxybenzenesulfonic acid or alkyloxybenzenesulfonic acid salt), and the second chamber contains an activating agent (e.g., an agent that activates the bleaching agent precursor or promotes or enhances the activity of the bleaching agent), for example, an aqueous solution or a peroxide source.
1.3. Package 1 or 1.1, wherein a first chamber contains a bleaching agent (e.g., a substantially anhydrous persulfate salt) or a bleaching agent precursor (e.g., an alkyloxybenzenesulfonic acid or alkyloxybenzenesulfonic acid salt), and the second chamber contains one or more ingredients incompatible with the bleaching agent (e.g., an aqueous solution) or bleaching agent precursor (e.g., a peroxide source).
1.4. Package 1.2, wherein the first chamber contains an alkyloxybenzenesulfonic acid or an alkyloxybenzenesulfonate salt and the second chamber contain a peroxide source, which upon mixing, react to form a peracid.
1.5. Package 1.4, wherein the first chamber contains an alkali metal alkyloxybenzenesulfonate salt, which reacts with the peroxide source to form an alkyl percarboxylic acid.
1.6. Package 1.5, wherein the alkali metal alkyloxybenzenesulfonate salt is sodium nonanyloxybenzenesulfonate, which reacts with the peroxide source to form pernonanoic acid.
1.7. Any of Packages 1.4-1.6, wherein the peroxide source is hydrogen peroxide, urea peroxide, carbamide peroxide, a percarbonate (e.g., sodium percarbonate), an alkyl hydrogen peroxide, or an alkali or alkaline earth metal peroxide (e.g., lithium peroxide, sodium peroxide),
1.8. Package 1.7, wherein the peroxide source is hydrogen peroxide (e.g., a 1-30% w/w aqueous solution of hydrogen peroxide, a 1-20%, or 1-10%, or 1-5%, or 2-3% w/w aqueous solution of hydrogen peroxide).
1.9. Package 1.3, wherein the first chamber contains a substantially anhydrous persulfate salt and the second chamber contains an aqueous solution.
1.10. Package 1.3, wherein the first chamber contains a solid persulfate salt (e.g., a powder) and the second chamber contains an aqueous solution.
1.11. Package 1.3 wherein the first chamber contains an aqueous solution or gel of a persulfate salt at a pH of 2-4, e.g., pH 2-3 or e.g., pH 3-4.
1.12. Any of Packages 1.9-1.11, wherein the persulfate salt comprises potassium peroxymonosulfate,
1.13. Package 1.12, wherein the persulfate salt comprises a mixture of potassium hydrogen peroxymonosulfate (KHSO₅), potassium hydrogen sulfate (KHSO₄) and potassium sulfate (K₂SO₄).
1.14. Package 1.13, wherein the persulfate salt comprises the 2 parts potassium hydrogen peroxymonosulfate to one part potassium hydrogen sulfate to one part potassium sulfate (2KHSO₅-KHSO₄-K₂SO₄).
1:15. Any of Packages 1.9-1.14, wherein the potassium hydrogen peroxymonosulfate (KHSO5) is present in the first chamber at a concentration of 2-90% by weight, e.g., from 10-80% by weight, or from 20-80% by weight, or from 30-80% by weight, or from 4-80% by weight, or from 50-80% by weight.
1.16. Any of Packages 1.9-1.14, wherein the second chamber contains an aqueous solution or gel at a pH of 4.5-8.5, e.g., pH 5-8, or, e.g., pH 6-8, or e.g., pH 6-7.
1.17. Any of Packages 1.9-1.14, wherein the second chamber contains a basic aqueous solution or gel such that upon mixing of the contents of the two chambers, the active composition has a pH of 4.5-8.5, e.g., pH 5-8, or, e.g., pH 6-8, or e.g., pH 6-7.
1:18. Any of the foregoing packages, further comprising an alkali metal tripolyphosphate (e.g., sodium tripolyphosphate or potassium tripolyphosphate) and/or an alkali metal pyrophosphate (e.g., sodium pyrophosphate or potassium pyrophosphate), and/or an alkali metal phosphate (e.g., any mono-, di- or tri-basic sodium or potassium phosphate, for example, trisodium phosphate).
1.19. Any of the foregoing packages wherein one or more of the chambers contains a gel.
1.20. Any of the foregoing packages wherein one or more of the chambers contains an aqueous solution.
1.21. Any of the foregoing packages wherein at least two chambers contain a gel, or wherein at least two chambers contain an aqueous solution.
1.22. Any of the foregoing packages, wherein at least one chamber contains a solid (e.g. a powder) and the other chamber contains an aqueous solution.
1.23. Any of the foregoing packages, wherein at least one chamber contains an anhydrous hydrophobic whitening composition (e.g., containing a monoperoxysulfate salt), such as a hydrophobic gel, and at least one other chamber contains a hydrophilic gel, e.g., a gel containing from 5 to 90 wt % water and one or more hydrophilic polymers, for example a gellant (e.g., a carbomer).
1.24. Any of the foregoing packages wherein one of the chambers contains a low viscosity aqueous solution and another contains a gellant, such that upon mixing and formation of the active bleaching agent, an extrudable gel is formed by the liquid and the gellant, which extrudable gel can then be extruded and applied to the teeth, e.g., using a tray or strip, for sufficient time, e.g., 10-30 minutes, to allow the teeth to bleach.
1.25. Any of the foregoing packages wherein upon mixing of the contents of the chambers, an extrudable gel is formed that can be applied to the teeth, e.g., using a tray or strip, for sufficient time, e.g., 10-30 minutes, to allow the teeth to bleach.
1.26. Any of the foregoing packages wherein an applicator is provided that can be used to mix the components and/or to apply the active composition to the teeth.
1.27. Any of the foregoing packages wherein the deformable material is plastic or aluminum.
1.28. Any of the foregoing packages wherein at least one chamber contains a low viscosity liquid solution having a viscosity sufficiently low to ensure efficient mixing with the contents of the other chamber or chambers, e.g., below 5,000 cps, e.g. below 500 cps.
1.29. Any of the foregoing packages wherein at least one chamber comprises a buffer.
1.30. Any of the foregoing packages wherein the ingredients of the chambers are present in amounts sufficient to provide, upon mixing, a bleaching agent in an amount and concentration effective to whiten teeth.
1.31. Any of the foregoing packages wherein one chamber contains a liquid (e.g., an aqueous solution) and a second chamber contains a gellant in powder form.
1.32. The foregoing package wherein the gellant is selected from carbomer gellants (e.g., Carbopol 971P), polysaccharide gums, such as xanthan gum, modified food starches, animal or fish-based gelatin, and silicas.
1.33. The foregoing package wherein the gellant is a carbomer gellant.
1.34. Any of the foregoing packages wherein the second chamber contains a gellant in powder form in a relative amount to provide a viscosity of 100,000 to 150,000 cps, e.g., about 125,000 cps, upon mixing with the contents of the first chamber, e.g., wherein the gellant is present in amount of from 5% to 50% by weight of the final active composition.
1.35. Any of the foregoing packages which further comprises an applicator device such as a dental tray or strip for applying a mixture of the contents of the first and second chambers to the teeth.
1.36. The foregoing package wherein, when the mixture is dispensed, the opening from the second chamber is directly attached to a tray so that the mixture is extruded into the tray.
1.37. Any of the foregoing packages wherein the mixture of the contents of the chambers is orally acceptable.
1.38. Any of the foregoing packages wherein one or more of the chambers comprises a flavoring.
1.39. Any of the foregoing packages wherein one or more of the chambers comprises a polymer which adheres to the tooth and/or gum surface.
1.40. Any of the foregoing packages, wherein one or more of the chambers comprise a surfactant (e.g., a detergent, emulsifier, penetrant or wetting agent), for example, sodium lauryl sulfate, an ethylene oxide/propylene oxide block copolymer, a polyoxyethylene sorbitan monolaurate, or sodium dodecylbenzenesulfonate.
1.41 Any of the foregoing packages, wherein one or more of the chambers comprise a whitening enhancer, such as ammonium persulfate, sodium persulfate or potassium persulfate.
1.42. Any of the foregoing packages, wherein one or more of the chambers comprise a desensitizing agent, for example, a potassium salt (e.g., potassium chloride or potassium nitrate) or a strontium salt (e.g., strontium chloride).
1.43. Any of the foregoing packages, wherein one or more of the chambers further comprise a humectant, for example a polyol, such as propylene glycol, glycerol, sorbitol, erythritol or xylitol.
1.44. Any of the foregoing packages, wherein one or more of the chambers further comprise a fluoride source, for example, sodium fluoride, potassium fluoride, stannous fluoride, zinc fluoride, or sodium monofluorophosphate.
1.45. Any of the foregoing packages, wherein one or more of the chambers further comprises an additional bleaching agent, for example, a peroxy compound, a chlorite, a hypochlorite, a percarbonate, a perborate, a persilicate, or a perphosphate.
1.46. Any of the foregoing packages, wherein the package includes an applicator, for example, an applicator that can be used to break the frangible barrier between the chambers, or an applicator that may help apply the whitening composition, or an applicator that serves both functions.
1.47. Package 1.46, wherein the applicator comprises one or more tips selected from a foam tip, a brush tip, a swab tip, or any other suitable tip that is useful for applying the whitening formulation to the teeth (e.g., useful for applying the whitening formulation to hard-to-reach areas of the teeth).
1.48. Package 1.46 or 1.47, wherein the one or more tips of the applicator are each independently from 0.1 mm to 2.0 mm in diameter, e.g., 0.2 to 1.5 mm, or 0.4 to 1.3 mm, or about 0.2 mm, about 0.4 mm, about 0.6 mm, about 0.7 mm, about 0.8 mm, about 0.9 mm, about 1.0 mm, about 1.1 mm, about 1.2 mm, or about 1.4 mm in diameter.
1.49. Any of Packages 1.46-1.48, wherein the one or more tips of the applicator are each independently straight, bent, or angled, and/or are each independently of cylindrical or conical cross section (e.g., tapered conical), or triangular cross-section.
1.50. Any of packages 1.46-1.49 where the applicator has two functional ends of similar or non-similar design (e.g., where the two tips have different shapes, dimensions, or purpose).
1.51. Any foregoing package, wherein the outlet through which the active composition can be dispensed further comprises a mixing tip.
1.52. Any foregoing package, wherein at least one of the chambers comprises a deformable material such that manual or mechanical squeezing of the chamber will reduce its volume forcing the contents of the chamber into a second chamber, or into a mixing chamber, or into an exit channel, nozzle or other dispensing means.
1.53. Any of packages 1-1.52, wherein all chambers comprise non-deformable materials (e.g., glass, hard plastics such as polyethylene, polypropylene, polycarbonate), such that a means other than squeezing the package is used to mix and/or expel the contents of the two or more chambers.
1.54. Package 1.53, wherein the package is a multi-chamber syringe, e.g., a two or three chamber syringe, such that when the plunger of the syringe is depressed the contents of each chamber are extruded for mixing and dispensing, optionally through a common nozzle or mixing tip.
2. Package 2, a multiple-use package comprising a material (e.g., a deformable or non-deformable material) configured to form at least two sealed chambers, the package having a first chamber and a second chamber, and optionally additional chambers, wherein each chamber has an exit channel and the two or more exit channels are adjacent and positioned at a common neck to eject their contents into a mixing tip when the two chambers are caused to expel their components (e.g., by squeezing the chambers or by puncturing a barrier or by removing the barrier between the chambers), and wherein the contents of the two or more chambers are incompatible with each for storage, and upon mixing the contents of the chambers mix and interact (e.g. chemically react) to form an active tooth-whitening composition.
2.1. Package 2 wherein at least one first chamber contains a bleaching agent (e.g., a substantially anhydrous persulfate salt) or bleaching agent precursor (e.g., an alkyloxybenzenesulfonic acid or alkyloxybenzenesulfonic acid salt), and at least one second chamber contains an activating agent, e.g., an agent that activates a bleaching agent precursor or promotes or enhances the activity of a bleaching agent, for example, an aqueous solution or a peroxide source, or one or more ingredients incompatible with the contents of the first chamber, e.g., an aqueous solution or a peroxide source.
2.2. Package 2 or 2.1, wherein a first chamber a bleaching agent (e.g., a substantially anhydrous persulfate salt) or bleaching agent precursor (e.g., an alkyloxybenzenesulfonic acid or alkyloxybenzenesulfonic acid salt), and the second chamber contains an activating agent (e.g., an agent that activates the bleaching agent precursor or promotes or enhances the activity of the bleaching agent), for example, an aqueous solution or a peroxide source.
2.3. Package 2 or 2.1, wherein a first chamber contains a bleaching agent (e.g., a substantially anhydrous persulfate salt) or a bleaching agent precursor (e.g., an alkyloxybenzenesulfonic acid or alkyloxybenzenesulfonic acid salt), and the second chamber contains one or more ingredients incompatible with the bleaching agent (e.g., an aqueous solution) or bleaching agent precursor (e.g., a peroxide source).
2.4. Package 2.2, wherein the first chamber contains an alkyloxybenzenesulfonic acid or an alkyloxybenzenesulfonate salt and the second chamber contain a peroxide source, which upon mixing react to form a peracid.
2.5. Package 2.4, wherein the first chamber contains an alkali metal alkyloxybenzenesulfonate salt, which reacts with the peroxide source to form an alkyl percarboxylic acid.
2.6. Package 2.5, wherein the alkali metal alkyloxybenzenesulfonate salt is sodium nonanyloxybenzenesulfonate, which reacts with the peroxide source to form pernonanoic acid.
2.7. Any of Packages 2.4-2.6, wherein the peroxide source is hydrogen peroxide, urea peroxide, carbamide peroxide, a percarbonate (e.g., sodium percarbonate), an alkyl hydrogen peroxide, or an alkali or alkaline earth metal peroxide (e.g., lithium peroxide, sodium peroxide).
2.8. Package 2.7, wherein the peroxide source is hydrogen peroxide (e.g., a 1-30% w/w aqueous solution of hydrogen peroxide, e.g., a 1-20%, or 1-10%, or 1-5%, or 2-3% w/w aqueous solution of hydrogen peroxide).
2.9. Package 2.3, wherein the first chamber contains a substantially anhydrous persulfate salt and the second chamber contains an aqueous solution.
2.10. Package 2.3 wherein the first chamber contains an aqueous solution or gel of a persulfate salt at a pH of 2-4, e.g., pH 2-3 or e.g., pH 3-4.
2.11. Package 2.9 or 2.10, wherein the persulfate salt comprises potassium peroxymonosulfate,
2.12. Package 2.11, wherein the persulfate salt comprises a mixture of potassium hydrogen peroxymonosulfate (KHSO₅), potassium hydrogen sulfate (KHSO₄) and potassium sulfate (K₂SO₄).
2.13. Package 2.12, wherein the persulfate salt comprises the 2 parts potassium hydrogen peroxymonosulfate to one part potassium hydrogen sulfate to one part potassium sulfate (2KHSO₅-KHSO₄-K₂SO₄).
2.14. Any of Packages 2.9-2 13, wherein the potassium hydrogen peroxymonosulfate (KHSO5) is present in the first chamber at a concentration of 2-90% by weight, e.g., from 10-80% by weight, or from 20-80% by weight, or from 30-80% by weight, or from 4-80% by weight, or from 50-80% by weight.
2.15. Any of Packages 2.9-2.14, wherein the second chamber contains an aqueous solution or gel at a pH of 4.5-8.5, e.g., pH 5-8, or, e.g., pH 6-8, or e.g., pH 6-7.
2.16. Any of Packages 2.9-2.14, wherein the second chamber contains a basic aqueous solution or gel such that upon mixing of the contents of the two chambers, the active composition has a pH of 4.5-8.5, e.g., pH 5-8, or, e.g., pH 6-8, or e.g., pH 6-7.
2.17. Any of the foregoing packages, further comprising an alkali metal tripolyphosphate sodium tripolyphosphate or potassium tripolyphosphate) and/or an alkali metal pyrophosphate (e.g., sodium pyrophosphate or potassium pyrophosphate), and/or an alkali metal phosphate (e.g., any mono-, di- or tri-basic sodium or potassium phosphate, for example, trisodium phosphate).
2.18. Any of the foregoing packages wherein one or more of the chambers contains a gel.
2.19. Any of the foregoing packages wherein one or more of the chambers contains an aqueous solution.
2.20. Any of the foregoing packages wherein at least two chambers contain a gel, or wherein at least two chambers contain an aqueous solution.
2.21. Any of the foregoing packages, wherein at least one chamber contains an anhydrous hydrophobic whitening composition (e.g., containing a monoperoxysulfate salt), such as a hydrophobic gel, and at least one other chamber contains a hydrophilic gel, e.g., a gel containing from 5 to 90 wt % water and one or more hydrophilic polymers, for example a gellant e.g., a carbomer).
2.22. Any of the foregoing packages wherein one of the chambers contains a low viscosity aqueous solution and another contains a gellant, such that upon mixing and formation of the active bleaching agent, an extrudable gel is formed by the liquid and the gellant, which extrudable gel can then be extruded and applied to the teeth, e.g., using a tray or strip, for sufficient time, e.g., 10-30 minutes, to allow the teeth to bleach.
2.23. Any of the foregoing packages, wherein the mixing tip is attached to the neck of the exit channel and is removable (for example, the mixing tip attaches to the neck via a screw-thread, or a slip tip).
2.24. The foregoing package, wherein the neck of the exit channel is covered with a frangible barrier that is broken up first use, or is covered by a removable cap or plug.
2.25. Any of the foregoing packages wherein upon mixing of the contents of the chambers, an extrudable gel is formed that can be applied to the teeth, e.g., using a tray or strip, for sufficient time, e.g., 10-30 minutes, to allow the teeth to bleach.
2.26. Any of the foregoing packages wherein an applicator is provided that can be used to apply the active composition to the teeth.
2.27. Any of the foregoing packages wherein the deformable material is plastic or aluminum.
2.28. Any of the foregoing packages wherein at least one chamber contains a low viscosity liquid solution having a viscosity sufficiently low to ensure efficient mixing with the contents of the other chamber or chambers, e.g., below 5,000 cps, e.g. below 500 cps.
2.29. Any of the foregoing packages wherein at least one chamber comprises a buffer.
2.30. Any of the foregoing packages wherein the ingredients of the chambers are present in amounts sufficient to provide, upon mixing, a bleaching agent in an amount and concentration effective to whiten teeth.
2.31. Any of the foregoing packages wherein one chamber contains a liquid (e.g., an aqueous solution) and a second chamber contains a gellant in powder form.
2.32. The foregoing package wherein the gellant is selected from carbomer gellants (e.g., Carbopol 971P), polysaccharide gums, such as xanthan gum, modified food starches, animal or fish-based gelatin, and silicas.
2.33. The foregoing package wherein the gellant is a carbomer gellant.
2.34. Any of the foregoing packages wherein the second chamber contains a gellant in powder form in a relative amount to provide a viscosity of 100,000 to 150,000 cps, e.g., about 125,000 cps, upon mixing with the contents of the first chamber, e.g., wherein the gellant is present in amount of from 5% to 50% by weight of the final active composition.
2.35. Any of the foregoing packages which further comprises an applicator device such as a dental tray or strip for applying a mixture of the contents of the first and second chambers to the teeth.
2.36. The foregoing package wherein, when the mixture is dispensed, the opening from the mixing tip is directly attached to a tray so that the mixture is extruded into the tray.
2.37. Any of the foregoing packages wherein the mixture of the contents of the chambers is orally acceptable.
2.38. Any of the foregoing packages wherein one or more of the chambers comprises a flavoring.
2.39. Any of the foregoing packages wherein one or more of the chambers comprises a polymer which adheres to the tooth and/or gum surface.
2.40. Any of the foregoing packages, wherein one or more of the chambers comprise a surfactant (e.g., a detergent, emulsifier, penetrant or wetting agent), for example, sodium lauryl sulfate, an ethylene oxide/propylene oxide block copolymer, a polyoxyethylene sorbitan monolaurate, or sodium dodecylbenzenesulfonate.
2.41. Any of the foregoing packages, wherein one or more of the chambers comprise a whitening enhancer, such as ammonium persulfate, sodium persulfate or potassium persulfate.
2.42. Any of the foregoing packages, wherein one or more of the chambers comprise a desensitizing agent, for example, a potassium salt (e.g., potassium chloride or potassium nitrate) or a strontium salt (e.g., strontium chloride).
2.43. Any of the foregoing packages, wherein one or more of the chambers further comprise a humectant, for example a polyol, such as propylene glycol, glycerol, sorbitol, erythritol or xylitol.
2.44. Any of the foregoing packages, wherein one or more of the chambers further comprise a fluoride source, for example, sodium fluoride, potassium fluoride, stannous fluoride, zinc fluoride, or sodium monofluorophosphate,
2.45. Any of the foregoing packages, wherein one or more of the chambers further comprises an additional bleaching agent, for example, a peroxy compound, a chlorite, a hypochlorite, a percarbonate, a perborate, a persilicate, or a perphosphate.
2.46. Any of the foregoing packages, wherein the package includes an applicator, for example, an applicator that may help apply the whitening composition to the teeth.
2.47. Package 2.46, wherein the applicator comprises one or more tips selected from a foam tip, a brush tip, a swab tip, or any other suitable tip that is useful for applying the whitening formulation to the teeth.
2.48. Package 2.16 or 2.47, wherein the one or more tips of the applicator are each independently from 0.1 mm to 2.0 mm in diameter, e.g., 0.2 to 1.5 mm, or 0.4 to 1.3 mm, or about 0.2 mm, about 0.4 mm, about 0.6 mm, about 0.7 mm, about 0.8 mm, about 0.9 mm, about 1.0 mm, about 1.1 mm, about 1.2 mm, or about 1.4 mm in diameter.
2.49. Any of Packages 2.46-2.48, wherein the one or more tips of the applicator are each independently straight, bent, or angled, and/or are each independently of cylindrical or conical cross section (e.g., tapered conical), or triangular cross-section.
2.50. Any of packages 2.46-2.49 where the applicator has two functional ends of similar or non-similar design (e.g., where the two tips have different shapes or dimensions).
2.51. Any foregoing package, wherein at least one of the chambers comprises a deformable material such that manual or mechanical squeezing of the chamber will reduce its volume forcing the contents of the chamber into a mixing chamber, or into an exit channel, nozzle or other dispensing means,
2.52. Any of packages 2-2.50, wherein all chambers comprise non-deformable materials (e.g., glass, or hard plastics such as polyethylene, polypropylene, polycarbonate), such that a means other than squeezing the package is used to mix and/or expel the contents of the two or more chambers.
2.53. Package 2.52, wherein the package is a multi-chamber syringe, e.g., a two or three chamber syringe, such that when the plunger of the syringe is depressed the contents of each chamber are extruded for mixing and dispensing, optionally through a common nozzle or mixing tip.
3. A method (Method 3) of whitening teeth comprising activating a multi-component, single-use or multiple-use oral care composition from any of Package 1, et seq., or Package 2, et seq., by combining the two or more components, and applying an effective amount of the mixture thus obtained to the teeth, e.g., using an applicator, for example a dental tray or a strip, for a sufficient time, e.g., at least 10 minutes, for example 10-30 minutes, to whiten the teeth.
3.1. Method 3, further comprising more than one treatment period each lasting, for example from 10 to 30 minutes, for example, four or five treatment periods.

An example of a particularly useful agent for use in the present disclosure is a triple salt mixture comprising potassium hydrogen peroxymonosulfate, potassium hydrogen sulfate, and potassium sulfate. Optionally, such a mixture may further include potassium peroxydilsulfate. An example of such a commercially available mixture is “Oxone”, which is the trade name of a mixture sold by DuPont. Oxone consists of 43% potassium hydrogen peroxymonosulfate, 23% potassium hydrogen sulfate, 29% potassium sulfate, 3% potassium peroxidisulfate, and 2% magnesium carbonate. Mixtures of these potassium reagents are usually available as a powder or solid which, when dissolved in water, typically forms a highly acidic solution (e.g., pH 1-4) which is fairly stable on storage. For example, a 1-3% solution of Oxone has a pH of 2.0-2.3. Above pH 6, however, these mixtures are strong oxidizing agents which readily decomposes to release reactive oxygen species. Many sources uses the terms “potassium hydrogen peroxymonosulfate” or “potassium peroxymonosulfate” to refer to the above triple salt mixture that comprises Oxone (2KHSO₅-KHSO₄-K₂SO₄). As used herein, however, terms “potassium hydrogen peroxymonosulfate” and “potassium peroxymonosulfate” refer to the individual chemical species with the formula KHSO₅.

As noted above, the invention may comprise gellants, for example carbomer gellants (e.g., Carbopol 971P), polysaccharide gums, such as xanthan gum, modified food starches, animal or fish-based gelatin, and silicas. Adhesive gel formulations for use with tooth whitening g agents are known in the art, e.g. as described in U.S. Pat. Nos. 7,862,801; 5,746,598; 6,730,316; 7,128,899. The gellant is useful to thicken whitening solutions to a point where they will not run out of a dental tray or away from the teeth to soft tissue areas. This allows the bleaching agent to stay in contact with the teeth for extended periods of time and protects soft tissues. The use of a dental tray and a viscous bleach allows a low concentration bleach to effectively whiten a person's teeth over a 1-2 week period of time with minimal risk to the patient. Gellants for this use should be selected and adjusted to provide a viscosity upon application of 100,000 to 150,000 cps, e.g., about 125,000 cps,

In a particular embodiment, the package or multi-part composition as hereinbefore described comprises a carbomer gellant, for example a modified polyacrylic acid hydrophilic polymer such as CARBOPOL® manufactured by Lubrizol. Carbomers are capable of forming viscous gels at concentrations above as little as 5% by weight. In some embodiments, one chamber contains a gellant and in other embodiments more than one chamber contains a gellant (e.g., both chambers).

In some embodiments, the contents of one or more of the chambers comprise a thickener and/or gelling agent at total levels of from 0.1 to 15% by weight, or from 0.4 to 10% by weight of the composition. Examples of thickeners and gelling agents useful herein include inorganic thickening silicas such as: an amorphous silica, for example Zeodent® 165 (Huber Corporation); Irish moss; iota-carrageenan; gum tragacanth; or polyvinylpyrrolidone, thickening agent selected from carboxyvinyl polymers, carrageenan, hydroxyethyl cellulose, laponite, water soluble salts of cellulose ethers such as sodium carboxymethylcellulose and sodium carboxymethyl hydroxyethyl cellulose, natural gums such as gum karaya, xanthan gum, gum arabic, and gum tragacanth, homopolymers of acrylic acid cross-linked with an alkyl ether of pentaerythritol or an alkyl ether of sucrose; carbomers, and combinations thereof. Particularly useful thickening agents include copolymers of lactide and glycolide monomers having the molecular weight in the range of from about 1,000 to about 120,000 (number average), and cellulose derivatives for example carboxymethyl cellulose), polysaccharide gums (for example xanthan gum or carrageenan gum), and combinations thereof.

Optional desensitizing agents include potassium salts, such as potassium citrate, potassium chloride, potassium tartrate, potassium bicarbonate, potassium oxalate, potassium nitrate, and strontium salts, and mixtures thereof.

One or more chambers may also include one or more surfactants. Suitable surfactants are those that are reasonably stable and foam throughout a wide pH range. The surfactant may be anionic, nonionic, amphoteric, zwitterionic, cationic, or mixtures thereof. Anionic surfactants useful herein include the water-soluble salts of alkyl sulfates having from 8 to 20 carbon atoms in the alkyl radical (e.g., sodium alkyl sulfate) and the water-soluble salts of sulfonated monoglycerides of fatty acids having from 8 to 20 carbon atoms. Sodium lauryl sulfate and sodium coconut monoglyceride sulfonates are examples of anionic surfactants of this type. Other suitable anionic surfactants are sarcosinates, such as sodium lauroyl sarcosinate, taurates, sodium lauryl sulfoacetate, sodium lauroyl isethionate, sodium laureth carboxylase, and sodium dodecyl benzenesulfonate. Mixtures of anionic surfactants can also be employed. Nonionic surfactants that can be used in the compositions can broadly be defined as compounds produced by the condensation of alkylene oxide groups (hydrophilic in nature) with an organic hydrophobic compound which may be aliphatic or alkyl-aromatic in nature. Examples of suitable nonionic surfactants include poloxamers (sold under trade name PLURONIC®), polyoxyethylene, polyoxyethylene sorbitan esters (sold under trade name TWEENS®), Polyoxyl 40 hydrogenated castor oil, fatty alcohol ethoxylates, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides, and mixtures of such materials. The amphoteric surfactants useful in the present invention can be broadly described as derivatives of aliphatic secondary and tertiary amines in which the aliphatic radical can be a straight chain or branched and wherein one of the aliphatic substituents contains from 8 to 18 carbon atoms and one contains an anionic water-solubilizing group, e.g., carboxylate, sulfonate, sulfate, phosphate, or phosphonate. Other suitable amphoteric surfactants are betaines, specifically cocamidopropyl betaine. Mixtures of amphoteric surfactants can also be employed. Many of these suitable nonionic and amphoteric surfactants are disclosed by Gieske et al. in U.S. Pat. No. 4,051,234. The present composition typically comprises one or more surfactants each at a level of from 0.25% to 12%, preferably from 0.5% to 8%, and most preferably from 1% to 6%, by weight of the composition.

Other optional additives may be included. Among such optional additives, included are those provided in order to change appearance or aesthetic appeal, and/or to preserve the final product, and/or for taste/cosmetic appeal and/or as therapeutic and prophylactic ingredients for oral health, prevention or treatment of a condition or disorder of hard or soft tissue of the oral cavity, or the prevention or treatment of a physiological disorder or condition. These optional additives include sweeteners, flavoring agents and colorants.

Sweeteners include both natural and artificial sweeteners. Suitable sweeteners include water soluble sweetening agents such as monosaccharides, disaccharides and polysaccharides such as xylose, ribose, glucose (dextrose), mannose, galactose, fructose (levulose), sucrose (sugar), maltose, water soluble artificial sweeteners such as the soluble saccharin salts, i.e., sodium or calcium saccharin salts, sucralose, cyclamate salts, dipeptide based sweeteners, such as L-aspartic acid derived sweeteners, including L-aspartyl-L-phenylalanine methyl ester (aspartame). In general, the effective amount of sweetener is utilized to provide the level of sweetness desired for a particular composition, which will vary with the sweetener selected. This amount will normally be from 0.001 to 5%, by weight. In some embodiments, the sweetener is sodium saccharin and is present at a concentration of about 0.01%, by weight.

Flavoring agents include, but are not limited to, natural and artificial flavors. These flavorings may be chosen from synthetic flavor oils and flavoring aromatics, and/or oils, oleo resins and extracts derived from plants, leaves, flowers, fruits and so forth, and combinations thereof. Representative flavor oils include: spearmint oil, cinnamon oil, peppermint oil, clove oil, bay oil, thyme oil, cedar leaf oil, oil of nutmeg, oil of sage, and oil of bitter almonds. These flavoring agents can be used individually or in admixture. Commonly used flavors include mints such as peppermint, artificial vanilla, cinnamon derivatives, and various fruit flavors, whether employed individually or in admixture. Generally, any flavoring agent or food additive, such as those described in Chemicals Used in Food Processing, publication 1274 by the National Academy of Sciences, pages 63-258, may be used. Typically, flavoring agents, if included, are present at a concentration of from 0.01 to 1%, by weight. In some embodiments, the flavoring agent may be present at a concentration of about 0.2%, by weight.

Colorants, such as dyes, may be food color additives presently certified under the Food Drug & Cosmetic Act for use in food and ingested drugs, including dyes such as FD&C Red No. 3 (sodium salt of tetraiodofluorescein), Food Red 17, disodium salt of 6-hydroxy-{(2-methoxy-5-methyl-4-sulphophenyl)azo}-2-naphthalenesulfonic acid, Food Yellow 13, sodium salt of a mixture of the mono and disulphonic acids of quinophtalone or 2-(2-quinolyl) indanedione, FD&C Yellow No. 5 (sodium salt of 4-p-sulfophenylazo-1-p-sulfophenyl-5-hydroxypyrazole-3 carboxylic acid), FD&C Yellow No. 6 (sodium salt of p-sulfophenylazo-B-naphtol-6-monosulfonate), FD&C Green No. 3 (disodium salt of 4-{[4-(N-ethyl-p-sulfobenzylamino)-phenyl]-(4-hydroxy-2-sulfoniumphenyl)-methylene}-[1-(N-ethyl-N-p-sulfobenzyl)-DELTA-3,5-cycl-ohexadienimine], FD&C Blue No. 1 (disodium salt of dibenzyldiethyl-diamino-triphenylcarbinol trisulfonic acid anhydrite), FD&C Blue No. 2 (sodium salt of disulfonic acid of indigotin) and mixtures thereof in various proportions. Typically, colorants if included are present in very small quantities.

In some embodiments, the carrier comprises a humectant, such as glycerin, sorbitol or an alkylene glycol such as polyethylene glycol or propylene glycol. In some embodiments, the carrier comprises a humectant at a level of from about 10 to about 80% by weight, or about 20 to about 60% by weight of the composition.

In some embodiments, the package comprises a surfactant or mixture of surfactants. Surfactants among those useful herein include water-soluble salts of at least one higher fatty acid monoglyceride monosulfate, such as the sodium salt of the monosulfated monoglyceride of hydrogenated coconut oil fatty acids; cocamidopropyl betaine; a higher alkyl sulfate such as sodium lauryl sulfate; an alkyl aryl sulfonate such as sodium dodecyl benzene sulfonate; a higher alkyl sulfoacetate; sodium lauryl sulfoacetate; a higher fatty acid ester of 1,2-dihydroxy propane sulfonate; and a substantially saturated higher aliphatic acyl amides of a lower aliphatic amino carboxylic acid, such as those having 12 to 16 carbons in the fatty acid, alkyl or acyl radicals; and mixtures thereof. Amides can be, for example, N-lauroyl sarcosine, and the sodium, potassium, and ethanolamine salts of N-lauroyl, N-myristoyl, or N-palmitoyl sarcosine. In various embodiments, the surfactant is present at a concentration of from 0.3 to 5% by weight of composition, or 0.5 to 3% by weight of composition.

All ingredients for use in the formulations described herein should be orally acceptable. By “orally acceptable” as the term is used herein is meant an ingredient which is present in the formulation as described in an amount and form which does not render the formulation unsafe, unpalatable, or otherwise unsuitable for use in the oral cavity.

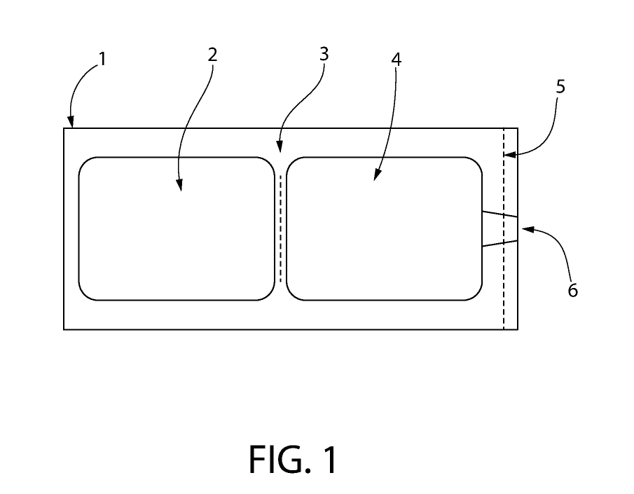

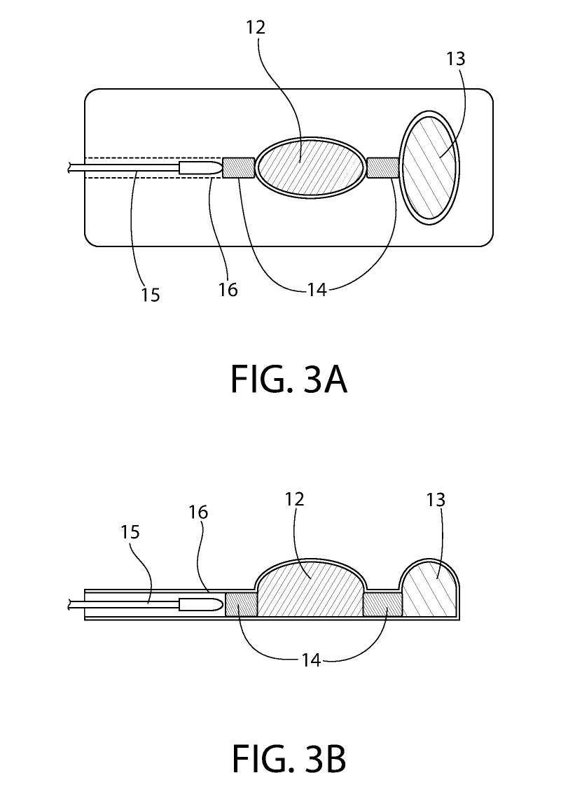

In some embodiments, the disclosure provides a package that is supplied with an applicator. The nature of the applicator may depend on the nature of the package (e.g., whether it is a package as shown in FIG. 1 or a package as shown in FIG. 3). The applicator may serve as a tool to puncture the frangible seal separating one or more chambers of the package, and may further serve to help mix the contents of the chambers. For example, after puncturing the seal, the consumer may push the brush back and forth through the chamber to help drag and mix the contents of the chamber, and furthermore, the consumer may rotate the applicator to further assist mixing. In addition, the applicator may be used to help apply the fully mixed composition to the teeth, and especially, to apply the mixture to hard-to-reach areas of the teeth. Such hard to reach areas may include, for example, the rear surfaces of the teeth, the margins of the teeth where adjacent teeth are in contact or nearly in contact, and the spaces between the teeth just above the gum line. For this latter purpose, the applicator may, for example, contain a foam tip, a brush tip, a swab tip, or any other suitable tip that is useful for applying the whitening formulation to the teeth. The applicator tip may range, for example, from 0.1 mm to 2.0 mm in diameter, e.g., 0.2 to 1.5 mm, or 0.4 to 1.3 mm, or about 0.2 mm, about 0.4 mm, about 0.6 mm, about 0.7 mm, about 0.8 mm, about 0.9 mm, about 1.0 mm, about 1.1 mm, about 1.2 mm, or about 1.4 mm in diameter. Furthermore, the applicator tip may be straight, bent, or angled, and may be of cylindrical or conical cross section (e.g., tapered conical, with a wider base that narrows toward the distal tip). The brush tip may also have a triangular shape (e.g., brush filaments positioned 60 degrees apart in cross-section), which would useful for applying whitening agent in the interdental region (the base between adjacent teeth). In a further embodiment, the applicator has two opposite functional ends, for example, one end is pointed and intended for puncturing the frangible seals and the other end is an applicator tip as described above for mixing and for applying the whitening composition. Alternatively, the two ends of the applicator may both be applicator tips as described above (e.g., where the two tips have different diameter, or one tip that is straight and the other is angled, or where the two tips have different shapes).

As used herein, “mixing tip” refers to any tip known to those skilled in the art designed to enable the mixing of a binary composition during extrusion through the tip. For example, such tips often include internal channels or guides, e.g., spiral channels or guides, which promote the mixing of the separated components such that upon exit from the mixing tip the two components are adequately mixed.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

1. A single-use package comprising a material configured to form at least two sealed chambers, the package having
(i) a first chamber, a second chamber, and optionally additional chambers, the chambers optionally separated by one or more barriers which are frangible or tearable, wherein the contents of the two chambers are incompatible with each for storage, wherein when one or more of the barriers are broken, the contents of the chambers mix and interact to form an active tooth-whitening composition; and
(ii) an opening means, for example a scored region, cap or plug, or one or more exit channels with a frangible barrier, cap or plug, which allows opening of the package, mixing of the components of the chambers and providing an outlet through which the active composition can be dispensed after mixing of the components;
wherein at least one first chamber contains a bleaching agent or bleaching agent precursor, and at least one second chamber contains an activating agent.

2. A multiple-use package comprising a material configured to form at least two sealed chambers, the package having
a first chamber and a second chamber, and optionally additional chambers, wherein each chamber has an exit channel and the two or more exit channels are adjacent and positioned at a common neck to eject their contents into a mixing tip when the two chambers are caused to expel their components, and wherein the contents of the two or more chambers are incompatible with each for storage, and upon mixing the contents of the chambers mix and interact to form an active tooth-whitening composition;
wherein at least one first chamber contains a bleaching agent or bleaching agent precursor, and at least one second chamber contains an activating agent.

3. The package of claim 1, wherein the first chamber contains an alkyloxybenzenesulfonic acid or an alkyloxybenzenesulfonate salt and the second chamber contain a peroxide source, which upon mixing reacts to form a peracid.

4. The package of claim 3 wherein the first chamber contains an alkali metal alkyloxybenzenesulfonate salt, which reacts with the peroxide source to form an alkyl percarboxylic acid.

5. The package of claim 3, wherein the alkali metal alkyloxybenzenesulfonate salt is sodium nonanyloxybenzenesulfonate, which reacts with the peroxide source to form pernonanoic acid.

6. The package of claim 3, wherein the peroxide source is hydrogen peroxide, urea peroxide, carbamide peroxide, a percarbonate, an alkyl hydrogen peroxide, or an alkali or alkaline earth metal peroxide.

7. The package of claim 6, wherein the peroxide source is hydrogen peroxide.

8. The package of claim 1, wherein the first chamber contains a substantially anhydrous persulfate salt or a solid persulfate salt, and the second chamber contains an aqueous solution.

9. The package of claim 8, wherein the first chamber contains an aqueous solution or gel of a persulfate salt at a pH of 2-4, e.g., pH 2-3 or e.g., pH 3-4.

10. The package of claim 8, wherein the persulfate salt comprises potassium peroxymonosulfate.

11. The package of claim 8, wherein the persulfate salt comprises a mixture of potassium hydrogen peroxymonosulfate (KHSO₅), potassium hydrogen sulfate (KHSO₄) and potassium sulfate (K₂SO₄).

12. The package of claim 11, wherein the persulfate salt comprises the 2 parts potassium hydrogen peroxymonosulfate to one part potassium hydrogen sulfate to one part potassium sulfate (2KHSO₅-KHSO₄-K₂SO₄).

13. The package of claim 8, wherein the second chamber contains an aqueous solution or gel at a pH of 4.5-8.5, e.g., pH 5-8, or, e.g., pH 6-8, or e.g., pH 6-7.

14. The package of claim 8, wherein the second chamber contains a basic aqueous solution or gel such that upon mixing of the contents of the two chambers, the active composition has a pH of 4.5-8.5, e.g., pH 5-8, or, e.g., pH 6-8, or e.g., pH 6-7.

15. The package of claim 1, wherein upon mixing of the contents of the chambers, an extrudable gel is formed.

16. The package of claim 1, wherein an applicator is provided that can be used to mix the components and/or to apply the active composition to the teeth.

17. The package of claim 1, wherein one or more of the chambers further comprises an additional bleaching agent selected from the group consisting of a peroxy compound, a chlorite, a hypochlorite, a percarbonate, a perborate, a persilicate, and a perphosphate.

18. The package of claim 1, wherein the package is a multi-chamber syringe.

19. A method of whitening teeth comprising activating a multi-component, single-use oral care composition from a package according to claim 1, by combining the two or more components, and applying an effective amount of the mixture thus obtained to the teeth.
